(19) United States
(12) Patent Application Publication
(10) Pub. No.: US 2012/0141491 A1
(43) Pub. Date: Jun. 7, 2012

(54) METHODS AND COMPOSITIONS FOR THE TREATMENT OF CANCERS AND PATHOGENIC INFECTIONS

(75) Inventors: William A. Dunn, Gainesville, FL (US); Debra Everton Akin, Micanopy, FL (US); Brian Keith Law, Gainesville, FL (US)

(73) Assignee: University of Florida Research Foundation Inc., Gainesville, FL (US)

(21) Appl. No.: 13/389,476

(22) PCT Filed: Aug. 9, 2010
(86) PCT No.: PCT/US10/44841

§ 371 Date: Feb. 8, 2012

Related U.S. Application Data
(60) Provisional application No. 61/232,816, filed on Aug. 11, 2009.

(51) Int. Cl. A61K 31/505; A61K 31/499; A61K 31/53; A61K 31/343; A61K 31/496; A61K 31/4965; A61K 31/5415; A61K 31/4035; A61K 31/395; A61K 31/4545; A61K 31/52; A61K 31/519; A61K 31/445; A61K 31/15; A61P 35/00; A61P 31/04; A61P 31/12; A61K 31/546; A61K 31/7052; A61K 31/65; A61K 38/43; A61K 33/24; A61K 39/395; C12N 5/02; G01N 33/53; A61K 31/145

(52) U.S. Cl. 424/141.1; 514/256; 514/649; 514/252.11; 514/244; 514/468; 514/252.13; 514/241; 514/255.06; 514/226.2; 514/416; 514/183; 514/316; 514/263.23; 514/267; 514/315; 514/639; 514/253.08; 514/206; 514/29; 514/154; 424/94.1; 424/649; 435/375; 435/7.1

(57) ABSTRACT

The subject application provides small compounds that are able to increase/enhance autophagy in various cells. These compounds are useful in augmenting existing treatments of various cancers, microbial/viral infections, and neurodegenerative diseases. Thus, the subject application also provides methods of treating various types of cancers, microbial/viral infections, and neurodegenerative diseases.

### CROSS-REFERENCE TO RELATED APPLICATION

This application claims the benefit of U.S. Provisional Application Ser. No. 61/232,816, filed Aug. 11, 2009, the disclosure of which is hereby incorporated by reference in its entirety, including all figures, tables and amino acid or nucleic acid sequences.

### BACKGROUND OF THE INVENTION

Autophagy is an essential cellular pathway for the degradation of macromolecules and recycling of anabolic precursors such as amino acids. This pathway is useful in the removal of protein aggregates present in a number of neurological disorders and is essential for the survival of breast carcinoma (e.g., MDA-MB468 and MDA-MB231), osteosarcoma (Saos-2), and hepatoma (HuH7) cell lines when deprived of amino acids and serum growth factors.

Cancerous tumor growth proceeds by stimulating protein synthesis and inhibiting protein degradation by autophagy. However, when autophagy is activated, sustained protein loss will exceed some threshold level causing cell death. In addition to routine housekeeping, autophagy also functions to protect the cell from damaged mitochondria that accumulate during aging and intracellular pathogens be they bacterial (e.g. Streptococcus pyogenes, Staphylococcus aureus, Listeria monocytogenes, Mycobacterium tuberculosis, Shigella flexneri, and Rickettsia conoril) or viral (e.g., Herpes) that can damage the host cell. Drugs that enhance autophagy will prevent the replication and survival of these pathogens within host cells by sequestering them for lysosomal destruction. Finally, enhancing autophagy may also be beneficial for the removal of protein aggregates that accumulate in neurodegenerative diseases such as Huntington's, Parkinson's, Charcot-Marie-Tooth type 1A, and Amyotrophic Lateral Sclerosis (ALS).

### BRIEF DESCRIPTION OF THE FIGURES

FIG. 1. Molecular docking sites for Bcl-2 and Bcl-XL. The BH3 domain/motif lies in Site 1. Site 2 is the aliphatic groove for binding Beclin-1 (a BH-1 domain/motif).

FIGS. 2A-2G. Activation of autophagy by Bcl-XL targeting compounds. The positions of C2 in Site 1 (FIG. 2A), C5 in Site 1 (FIG. 2B), and C8 in Site 2 (FIG. 2C) of Bcl-xl are shown by in silico molecular docking. Saos-2 osteosarcoma cells expressing GFP-LC3 were incubated in enriched (Fed) medium in the presence of C2 (FIG. 2D), C5 (FIG. 2E) and C8 (FIG. 2F) Bcl-targeted compounds. GFP-LC3 is found diffuse throughout the cytosol and nucleus when autophagy is suppressed. Ongoing autophagy can be visualized by the presence of GFP-LC3 at autophagic vacuoles (AVs) that appear as dots (arrows). Our observations conclude that C5 and C8, but not C2, enhanced autophagy. In FIG. 2E, Saos-2 osteosarcoma cells expressing GFP-LC3 were incubated in enriched (Fed) medium in the absence and presence of Bcl-targeted compounds or in medium lacking amino acids and serum (Starved). The small compounds (100 μM) ranged from ineffective (C1, C2, C6, C11 and C14) to those that stimulated autophagy to levels seen in amino acid and serum starved cells (C5, C8, C10 and C12; FIG. 2G).

FIGS. 3A-3E. Effects of Bcl-XL targeted small compounds on the autophagy response and cell survival in NeuT cells. The positions of C4 in Site 1 (FIG. 3A), and C9 in Site 2 (FIG. 3B) of Bcl-xl are shown by in silico molecular docking. NeuT (Her2-positive breast cancer cell line) cells expressing GFP-LC3 were incubated in enriched (Fed) medium in the presence of C4 (FIG. 3C) and C9 (FIG. 3D) Bcl-targeted compounds. GFP-LC3 is found on autophagic vacuole that appear as dots (arrows). In FIG. 3E, the survival of NeuT cells was quantified after 20h of treatment with 0.1-1000 μM of Site 1 (C2 and C4) and Site 2 (C7 and C9) compounds. C4 and C9 prove lethal to these cells, while C2 and C7 do not.

FIG. 4A-4F. Activation of autophagy by Bcl-targeted compounds. The localization of GFP-LC3 to autophagic vacuoles was enhanced by 100 mM Bcl-targeted compounds in Saos-2 osteosarcoma cells. The autophagy response in fed Saos2 cells was measured in the absence and presence of anti-Bcl compounds by visualizing the punctate localization of GFP-LC3 (FIGS. 4A-4C) and RFP-GFP-LC3 (FIGS. 4D-4F) to autophagic vacuoles. In untreated fed cells, the GFP-LC3 is localized uniformly throughout the cell. With GFP-LC3 (FIGS. 4A-4C), autophagic vacuoles are visualize as dots or ring-like structures of various sizes when cells were treated with compounds Bx-17 (FIG. 4B) or Bx-22 (FIG. 4C). Autophagy in cells expressing RFP-GFP-LC3 (FIGS. 4D-4F) was activated with compound B2-1. Structures containing RFP (red, FIG. 4D) and GFP (green, FIG. 4E) are autophagosomes (arrow), while RFP (red, FIG. 4D) structures are autolysosomes (arrowhead).

FIGS. 5A-5C. Characterization of proBeclin 1 compounds. Upon ranking 100 NCl compounds by in silico docking to Bcl-xl, the autophagy responses were evaluated in fed Saos-2 cells expressing GFP-LC3 (FIG. 5A). Those compounds that enhanced autophagy were then tested for their ability to increase protein degradation assessed by pulse-chase methods using 14C-valine (FIG. 5B). The rates of protein degradation (represented as the mean±SD of n=6) were found to be elevated in cells treated with Bcl-xl compounds (Bx-8 and Bx-17), Bcl-2 compounds (B2-1 and B2-7), and Bx-22, a compound that docks with both Bcl-xl and Bcl-2, when compared to a control compound, which has no effect on autophagy. Meanwhile, C11 (Bx-11), which promotes an accumulation of GFP-LC3 structures (FIG. 5A), suppressed protein degradation. As depicted in FIG. 5A, compounds with a black bar (Bx-8, Bx-17, B2-1, B2-7 and B2-11 (see Table 1)) stimulated proteolysis while those with a striped bar inhibited (Bx-11 and Bx-17 (see Table 1)). Finally, those compounds which enhanced autophagy and protein degradation were tested for there ability to interfere with Beclin 1/Bcl binding determined by immunoprecipitation using anti-Beclin 1 antibodies. Cells were maintained in nutrient-rich medium in the absence and presence of Bcl-targeted compounds for 4 hr. The cells were then solubilized, Beclin 1 complex immunoprecipitated, and the Beclin 1 hound proteins identified by Western blotting (FIG. 5C). The IgG-LC is the immunoglobin light chain of the anti-Beclin 1 antibody that was used to immunoprecipitate the complex. The absence of Bcl-xl is indicative of an inhibition of Beclin 1 binding. That is, B2-11 and Bx-8 inhibited Beclin 1 binding while Bx-16 and Bx-22 did not. Compounds with an asterisk (*) were found to interfere with binding while those with a pound (#) sign had no affect on Beclin 1 binding (FIG. 5A).

FIG. 6. Chemical structures of potential Bcl-targeted proBeclin 1 lead compounds that promote autophagy.

### DETAILED DISCLOSURE OF THE INVENTION

This application provides compositions and methods for improving the therapeutic efficacy of various cancer treatments, for example endocrine therapy, chemotherapy or radiation therapy, by enhancing macroautophagy, also referred to commonly as autophagy. Inhibition of Bcl-2 and/or Bcl-XL binding to Beclin-1, as provided by the subject invention, can activate autophagy thereby increasing the therapeutic efficacy of regimens commonly employed to treat cancer, such as for example, endocrine therapy, chemotherapy or radiation therapy. Thus, the methods provided in this application also provide for improving the efficacy of a cancer therapy, including endocrine therapeutic, chemotherapeutic or radiation treatments comprising the administration of compounds disclosed herein to a patient having cancer (see, for example, the compounds in Table 1 and Table 2 (providing chemical names and/or structures). In certain embodiments, compounds that are toxic to cells (capable of inducing cell death) may be preferred for use in the various methods of treating cancer or increasing the efficacy of cancer treatments as disclosed herein.

In the context of the subject application, the phrase “improving the efficacy” or “improving the therapeutic efficacy” refers to increasing the rate at which, or the degree to which, a cell responds to the cancer therapy, increasing or augmenting the effect of a cancer therapy on a cell, or increasing the likelihood that a cell will respond to the cancer therapy, as compared with the cellular response to the cancer therapy alone in the absence of the administration of compounds and compositions as disclosed herein.

The term “cell” refers to a single cell, a plurality of cells or a population of cells, unless otherwise indicated herein. The cell can be a cancerous cell, a cell that is suspected of being cancerous or pre-cancerous, or a cell that is pre-disposed to becoming cancerous or pre-cancerous. The cell may be a transformed cell or a cell undergoing abnormal or uncontrolled growth. The cell may be a cancerous cell with stem-cell like properties or a stem-cell with cancerous properties, for example uncontrolled proliferation or the capacity to differentiate into other cell types. However, in those embodiments in which bacterial or viral infections are being treated, the cell is, typically, not cancerous or malignant. The cell may be a cell in culture or it may be a cell within a subject. The cell may be derived from any organism whose cells undergo autophagy, and in particular embodiments is a mammalian cell, including a mouse cell, a rat cell, a rabbit cell or a human cell.

A cell that is currently undergoing cancer therapy refers to a cell that is currently being treated with a cancer therapy regimen, including simultaneously with, overlapping with, or sequentially prior to or following the administration of compounds or compositions that inhibit the interaction of Beclin-1 with Bcl-2 and/or Bcl-XL. In various aspects of the invention, the cancer therapy may be any one of a number of therapies given to a cell to treat, inhibit or prevent cancer and may, for example, include one or more therapies (e.g., chemotherapy, endocrine therapy, radiation therapy and/or chemoradiation therapy). Chemotherapy refers to treatment with drugs or chemical compounds that target cancer cells. Endocrine therapy, also called hormone therapy, refers to treatment that removes, blocks, or adds hormones. Radiation therapy refers to the use of high-energy radiation from x-rays, gamma rays, neutrons, protons and other sources to target cancer cells. Radiation may be administered externally or it may be administered using radioactive material given internally. Chemoradiation therapy combines chemotherapy and radiation therapy. In some aspects of the invention, the cancer therapy may have a cytotoxic or cytostatic effect. The cancer therapy may also be a therapy that invokes or induces cytoprotective autophagy in the cell.

The chemotherapy or endocrine therapy may involve, without limitation, administration of a chemotherapeutic agent or endocrine therapeutic agent comprising a small molecule, a peptide or a protein, an anthracycline, an alkylating agent, an alkyl sulfonate, an aziridine, an angiogenesis inhibitor, an ethylenimine, a methylmelamine, a nitrogen mustard, a nitrosourea, an antibiotic, an antimetabolite, a folic acid analogue, a purine analogue, a pyrimidine analogue, an enzyme, a podophyllotoxin, a platinum-containing agent or a cytokine. Preferably, the chemotherapeutic agent or endocrine therapeutic agent is one that is known to be effective against the particular cancer and cell type. Exemplary chemotherapeutic agents include taxanes, tamoxifen, cisplatin, adriamycin (ADR), 5-fluorouracil (5-FU), etoposide, doxorubicin, VEGF-TRAP or Avastin and variants thereof (see, for example, Proc. Natl. Acad. Sci., 2002, 99:11393-11398), AdPEDF (Adenovector Pigment Epithelium-Derived Factor; Retina, The Journal Of Retinal And Vitreous Diseases, 2005, 25 (No. 8):S48) small molecule tyrosine kinase inhibitors that target VEGFRs (such as cediranib (4-[(4-fluoro-2-methyl-1H-indol-5-yl)oxy]-6-methoxy-7-[3-(pyrrolidin-1-yl) propoxy]quinazoline), sunitinib (N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide), sorafenib (4-[4-[[4-chloro-3-(trifluoromethyl)phenyl]carbamoylamino]phenoxy]-N-methyl-pyridine-2-carboxamide) or pazopanib (benzenesulfonamide,5-[4-[(2,3-dimethyl-2H-indazol-6-yl)methylamino]-2-pyrimidinyl]-amino]-2-methyl-monohydrochloride)), thalidomide (2-(2,6-dioxopiperidin-3-yl)isoindoline-1,3-dione), lenalidomide (REVLIMID; 3-(4-amino-1-oxo-3H-isoindol-2-yl)piperidine-2,6-dione)), bevacizumab, HERCEPTIN (trastuzumab), or camptothecin.

The phrase “treating cancer” refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). Treating can also mean prolonging survival of a subject beyond that expected in the absence of treatment. In certain embodiments, cancers that are to be treated include, but are not limited to, breast cancer, pancreatic cancer, prostate cancer, gynecological cancers (e.g., ovarian cancer or cervical carcinoma), skin cancer (e.g., melanoma), brain cancer, neuroblastoma, glioma, a solid tumor, a hematologic malignancy (e.g., leukemia or lymphoma), head and cancer, ganglioneuroma, infiltrating ductal carcinoma of the breast, adenocarcinoma of the lung, pancreatic adenocarcinoma, pancreatic islet cell tumor, liver cancer, gastric cancer, bladder cancer, colon cancer, prostate cancer, lung cancer or nasopharyngeal carcinoma. Treating can also mean inhibiting the progression of disease, slowing the progression of disease temporarily, or halting the progression of the disease permanently. Additionally, individuals suitable for treatment as set forth herein can be a mammal, including humans, cats, dogs, horses, mice or rats.

In various aspects of the invention, compositions disclosed herein can be used in combination with a given cancer therapy and an effective amount of a composition as disclosed herein is administered to an individual. The term “effective amount” means an amount effective, at dosages and for periods of time necessary to achieve the desired result, for example, the inhibition of the interaction of Beclin-1 with Bcl-2 and/or Bcl-XL within the individual. Compositions containing the compounds disclosed herein may be administered to a subject using a variety of techniques. For example, the agent may be administered systemically, which includes by injection including intramuscularly or intravenously, orally, sublingually, transdermally, intraarterially, subcutaneously or internasally. Alternatively, the compositions may be administered directly at a site at which the cancer is located. Delivery to the site includes topical administration, injection to the site, or surgical implantation, for example at a site of a tumor.

Other embodiments of the subject invention provide for compositions comprising chemotherapeutic agents and the compounds disclosed herein for administration to an individual with cancer. Regardless of whether the compounds disclosed herein are administered alone or in combination with a chemotherapeutic agent, a pharmaceutically acceptable diluent can be used. The proportion and identity of the pharmaceutically acceptable diluent may be determined by the chosen route of administration, compatibility with live cells, and standard pharmaceutical practice. Generally, the pharmaceutical composition will be formulated with components that will not significantly impair the biological properties of the compounds disclosed herein.

The pharmaceutical composition can be prepared by methods for the preparation of pharmaceutically acceptable compositions suitable for administration to patients. Suitable vehicles are described, for example, in Remington's Pharmaceutical Sciences (Remington's Pharmaceutical Sciences, Mack Publishing Company, Easton, Pa., USA 1985).

As discussed above, a chemotherapy or endocrine therapy may involve, without limitation, administration of a chemotherapeutic agent or endocrine therapeutic agent comprising a small molecule, a peptide or a protein, an anthracycline, an alkylating agent, an alkyl sulfonate, an aziridine, an angiogenesis inhibitor, an ethylenimine, a methylmelamine, a nitrogen mustard, a nitrosourea, an antibiotic, an antimetabolite, a folic acid analogue, a purine analogue, a pyrimidine analogue, an enzyme, a podophyllotoxin, a platinum-containing agent or a cytokine in combination with a compound set forth in Table 1. Thus, compositions comprising one or more compound set forth in Table 1 in combination with a chemotherapeutic agent or endocrine therapeutic agent selected from taxanes, tamoxifen, cisplatin, Adriamycin (ADR), 5-fluorouracil (5-FU), etoposide, doxorubicin, bevacizumab, HERCEPTIN (generic name, trastuzumab) or camptothecin. Exemplary angiogenesis inhibitors suitable for use as disclosed herein include, bevacizumab, VEGF-TRAP or Avastin, and variants thereof (see, for example, Proc. Natl. Acad. Sci., 2002, 99:11393-11398)), AdPEDF (Adenovector Pigment Epithelium-Derived Factor; Retina, The Journal Of Retinal And Vitreous Diseases, 2005, 25 (No. 8):S48) small molecule tyrosine kinase inhibitors that target VEGFRs (such as cediranib (4-[(4-fluoro-2-methyl-1H-indol-5-yl)oxy]-6-methoxy-7-[3-(pyrrolidin-1-yl)propoxy]quinazoline), sunitinib (N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide), sorafenib (4-[4-[[4-chloro-3-(trifluoromethyl)phenyl]carbamoylamino]phenoxy]-N-methyl-pyridine-2-carboxamide) or pazopanib (benzenesulfonamide,5-[4-[(2,3-dimethyl-2H-indazol-6-yl)methylamino]-2-pyrimidinyl]-amino]-2-methyl-monohydrochloride)), thalidomide (2-(2,6-dioxopiperidin-3-yl)isoindoline-1,3-dione) and lenalidomide (REVLIMID; 3-(4-amino-1-oxo-3H-isoindol-2-yl)piperidine-2,6-dione)).

Once inside, bacterial and viral pathogens often modulate their cellular environment to establish an intracellular niche for survival and replication. Thus, another aspect of the invention provides methods for the treatment of pathogenic infections in an individual by enhancing the autophagic removal of such intracellular pathogens by the administration of a compound (or combination of compounds) disclosed in Table 1 alone or in combination with therapeutic agents suitable for the treatment of the pathogenic infection. Non-limiting examples of pathogens suitable for treatment via the administration of compounds and compositions disclosed herein include Streptococcus spp., Staphylococcus spp., Listeria spp., Mycobacterium spp., Shigella spp., Rickettsia spp. and various viral pathogens, such as herpes virus. In certain specific embodiments, Streptococcus pyogenes, Staphylococcus aureus, Listeria monocytogenes, Mycobacterium tuberculosis, Shigella flexneri, and/or Rickettsia conoril can be treated.

Thus, another aspect of the invention provides compositions comprising the compounds disclosed in Table 1 alone or in combination with a therapeutic agent suitable for the treatment of the pathogenic infection (e.g., antibiotics and/or antiviral agents). Non-limiting examples of antibiotics suitable for formulation with compounds as set forth in Table 1 include ciprofloxacin, norfloxacin, ofloxacin, ceftriaxone, azithromycin, nalidixic acid, ampicillin, a quinolone, a fluoroquinolone, tetracycline, a macrolide, a sulfa, clindamycin, penicillin, gentamycin, vancomycin, clindamycin, cefalexin, clarithyromycin, erythromycin, telithromycin, chlorampheni col, trimethoprim-sulfamethoxazole, rifamycin, isonazid, doxycycline, oxacillin, flucoxacillin, methicillin, cloxacillin, cephalosporin and carbapenems. Non-limiting examples of antiviral agents include: acyclovir, famciclovir and/or valaciclovir. Additional antibiotics and/or antiviral agents suitable for treating bacterial and viral infections that can also be combined with the compounds disclosed in Table 1.

Another aspect of the invention provides for a composition comprising a pharmaceutically acceptable diluent and a combination of the compounds disclosed in Table 1. Such compositions can be used for the manufacture of a medicament for the treatment of any of the cancers or pathogenic conditions discussed herein. As discussed above, compositions containing one or more compound as set forth in Table 1 can also contain a therapeutic or chemotherapeutic agent suitable for the treatment of cancer, bacterial infections or a viral infection.

A method of suppressing or inhibiting the interaction of Bcl-2 and/or Bcl-XL with Beclin-1 comprising contacting a sample containing Bcl-2 and/or Bcl-XL with a compound selected from those disclosed in Table 1 (or combinations of such compounds). In these methods, the samples can be contacted in vitro.

A method of enhancing or increasing the rate of degradation of protein aggregates within a cell by autophagy is also provided by the present disclosure. In this aspect of the invention, cells containing protein aggregates are contacted with a composition comprising a compound, such as that disclosed in Table 1. In certain aspects of the invention, compounds that are not toxic to cells are used to contact the cell (e.g., NSC 31331). This aspect of the invention is also suitable for the treatment of cells containing protein aggregates that accumulate in neurodegenerative diseases such as Huntington's disease, Parkinson's disease, Charcot-Marie-Tooth type 1A syndrome, and Amyotrophic Lateral Sclerosis (ALS). Thus, method of treating such diseases are also provided in which an individual having Huntington's disease, Parkinson's disease, Charcot-Marie-Tooth type 1 A syndrome or Amyotrophic Lateral Sclerosis (ALS) is administered a composition comprising a compound as provided in Table 1, in an amount sufficient to increase protein aggregate degradation within cells containing such aggregates. In this aspect of the invention, it may be preferred that compounds that are not toxic to cells, such as compound NSC 31331, be administered to an individual having one of the aforementioned conditions.

Another aspect of the invention provides a method of screening compounds that inhibit or reduce the formation of autophagic vacuoles comprising:

a) scoring compounds by in silico docking for the potential to bind to Bcl-2 and/or Bcl-XL within a first pocket that contains Q99, A100, G101, D102, D103, F104, and V148 of Bcl-2 and E92, A93, G94, D95, E96, F97, and V141 of Bcl-XL and/or a second pocket that contains V133, V134, L137, F138, N143, G145, R146, I147, and A149 of Bcl-2 and F105, D107, L108, L130, G138, R139, I140, A142, F143, F144, and 5145 of Bcl-XL;

b) selecting those compounds identified as having the potential to bind to pocket 1 and/or pocket 2 of Bcl-2 and/or Bcl-XL; and

c) testing the selected compounds for the ability to enhance autophagy, said testing comprising contacting cells with at least one of the selected compounds and assessing the ability of said at least one selected compound for the ability to increase autophagy activity, wherein an increase in the formation of autophagic vacuoles is indicative of a compound that increases autophagy in said cells.

In this aspect of the invention, compounds that can interact/bind to each individual domain/motif (also referred to as “a pocket” or “pockets”) are screened in silico and then tested in cellular based assays for the ability to increase or enhance autophagy. Various embodiments of the invention provide for the identification of compounds that bind to each individual pocket rather than a compound that interacts with both pockets of Bcl-XL and/or Bcl-2. Thus, one embodiment screens for compounds that interact with Site 1 (Q99, A100, G101, D102, D103, F104, and V148 of Bcl-2 and E92, A93, G94, D95, E96, F97, and V141 of Bcl-XL). Another embodiment screens for compounds that interact with Site 2 (V133, V134, L137, F138, N143, G145, R146, I147, and A149 of Bcl-2 and F105, D107, L108, L130, G138, R139, I140, A142, F143, F144, and S145 of Bcl-XL;). Another embodiment contemplates the identification of compounds that bind to both site pockets of Bcl-2 and/or Bcl-XL.

As discussed above, compounds selected on the basis of the in silico analysis are then screened for the ability to increase or enhance autophagy in cellular assays. One aspect of the invention provides for the testing/contacting of a cell expressing labeled microtubule-associated protein 1 light chain 3 beta, LC3, (Genebank NM_{—}022818, which is hereby incorporated by reference in its entirety) with a test compound and assessing the cells for the presence of the labeled LC3 at or in autophagic vacuole membranes. LC3 can be labeled with a variety of labels, including green fluorescent protein (GFP), or red fluorescent protein (RFP), or both GFP and RFP. In various embodiments, the cells can also contain protein aggregates, Streptococcus pyogenes, Staphylococcus aureus, Listeria monocytogenes, Mycobacterium tuberculosis, Shigella flexneri, Rickettsia conoril or herpes virus. Alternatively, the cells can be selected from a carcinoma, hepatoma, osteosarcoma or sarcoma cell line.

All patents, patent applications, provisional applications, and publications referred to or cited herein are incorporated by reference in their entirety, including all figures and tables, to the extent they are not inconsistent with the explicit teachings of this specification. Following are examples which illustrate procedures for practicing the invention. These examples should not be construed as limiting. All percentages are by weight and all solvent mixture proportions are by volume unless otherwise noted.

### Example 1

### Identification of Compounds that Inhibit Bcl-2/Bcl-Xl Interaction with Beclin-1

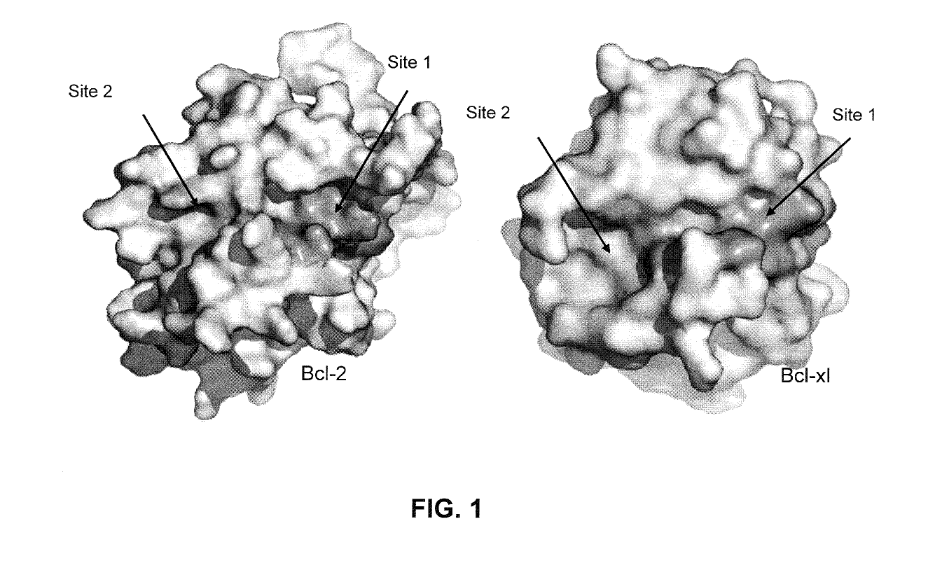

In silica molecular docking of Bcl targeted compounds: We have identified two surface “pockets” of Bcl-2 and Bcl-XL based on the crystal structures of Bcl-2 (PDB code 1GJH), Bcl-XL (PDB code 1R2D) and the Bcl-xl/Bcn-1 complex (PDB code 1P1L) from the Protein Data Bank. Site 1 pocket contains Q99, A100, G101, D102, D103, F104, and V148 of Bcl-2 and E92, A93, G94, D95, E96, F97, and V141 of Bcl-XL and Site 2 contains V133, V134, L137, F138, N143, G145, R146, I147, and A149 of Bcl-2 and F105, D107, L108, L130, G138, R139, I140, A142, F143, F144, and 5145 of Bcl-XL; (FIG. 1). The in silico molecular docking was performed using DOCK (v 5.2) software and a Linux PC Dual Quad-Core Xeon™ SAS PCI Express Graphics Workstation with 8 parallel processors. We set the software parameters to position each of 250,000 compounds from the NCI library in 1000 different orientations into the pocket. The program then ranked the top 500 compounds using a shape-scoring function (based on van der Waals attractive energy) and a function scoring electrostatic interactions.

### Example 2

### Autophagy in Presence of Candidate Compounds

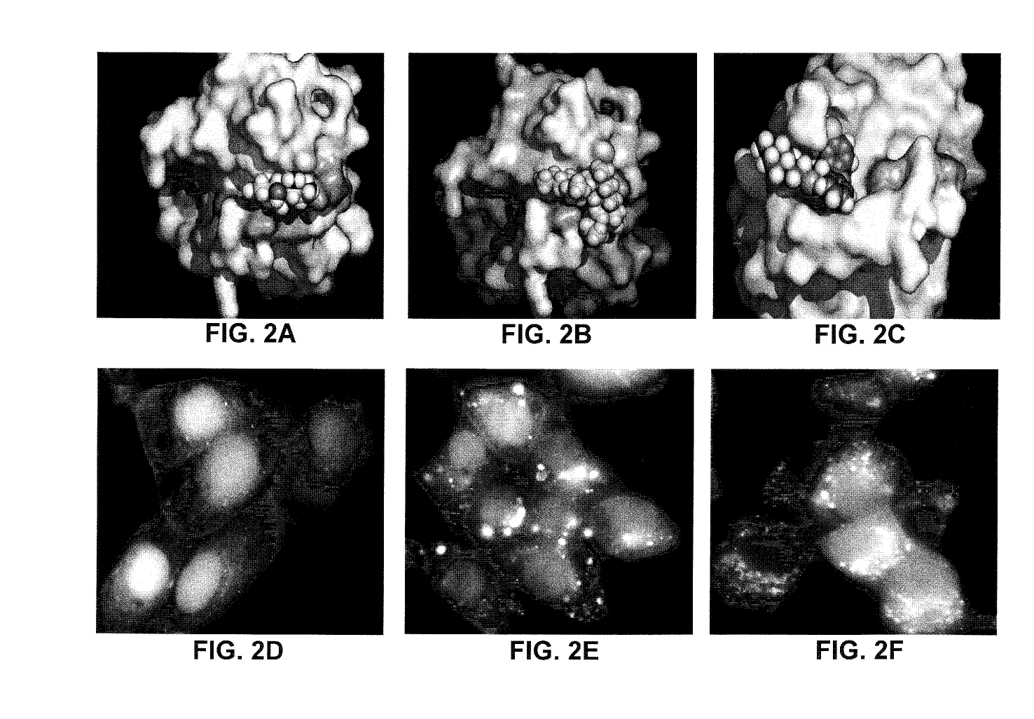

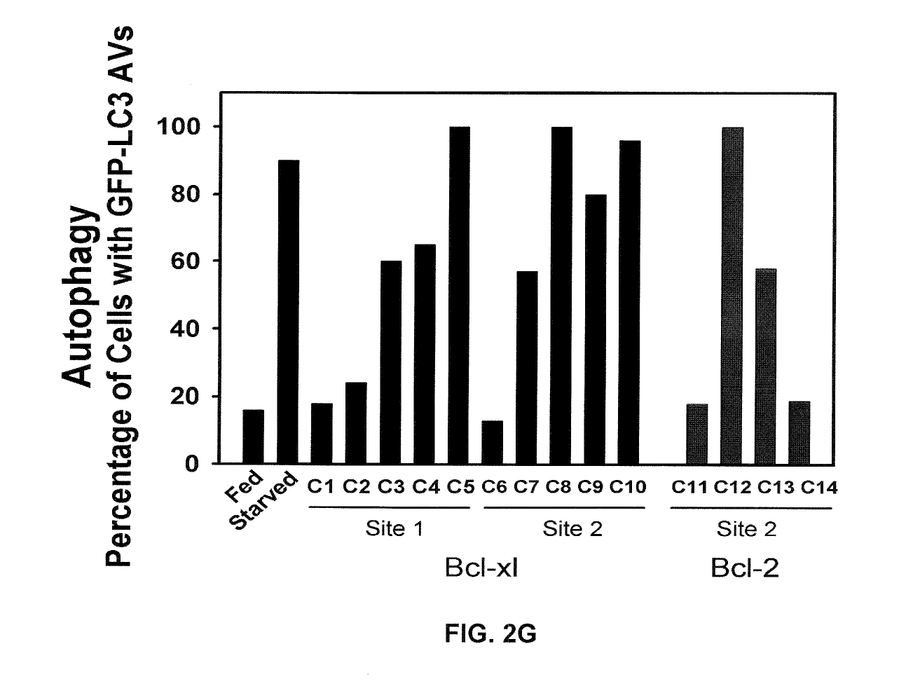

Bcl targeted compounds promote autophagy: The compounds identified by in silico molecular docking were tested for their ability to enhance autophagy (FIGS. 2A-G). This was done in Saos-2 osteosarcoma cells stably expressing the autophagosome marker, GFP-LC3. In fed cells not undergoing autophagy GFP-LC3 is a soluble protein found throughout the cytosol. In amino acid and serum starved cells, autophagy is activated and GFP-LC3 becomes associated with autophagic vacuoles that can be visualize by fluorescence microscopy. As illustrated in FIGS. 2D, 2E and 2F, GFP-LC3 distributes uniformly throughout the cytosol and nucleus when compounds such as C2 do not enhance autophagy, but found at autophagosomes (punctate pattern) when compounds such as C5 and C8 enhance autophagy. Of the 30 compounds tested thus far with more to be tested, three Bcl-xl site 1 (C3, C4, and C5), four Bcl-xl site 2 (C7, C8, C9, and C10), and two Bcl-2 site 2 compounds (C2 and C3) were found to promote autophagy (FIG. 2G).

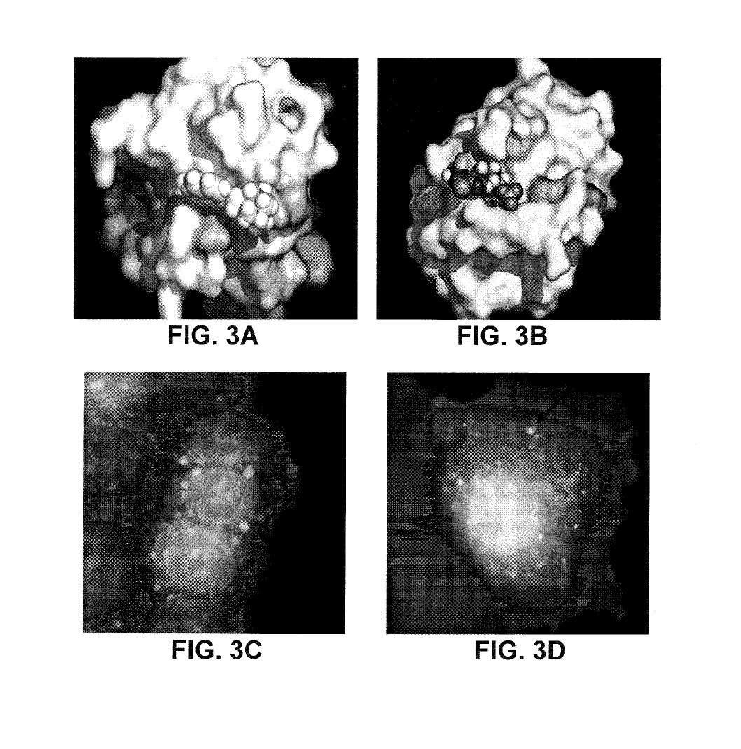

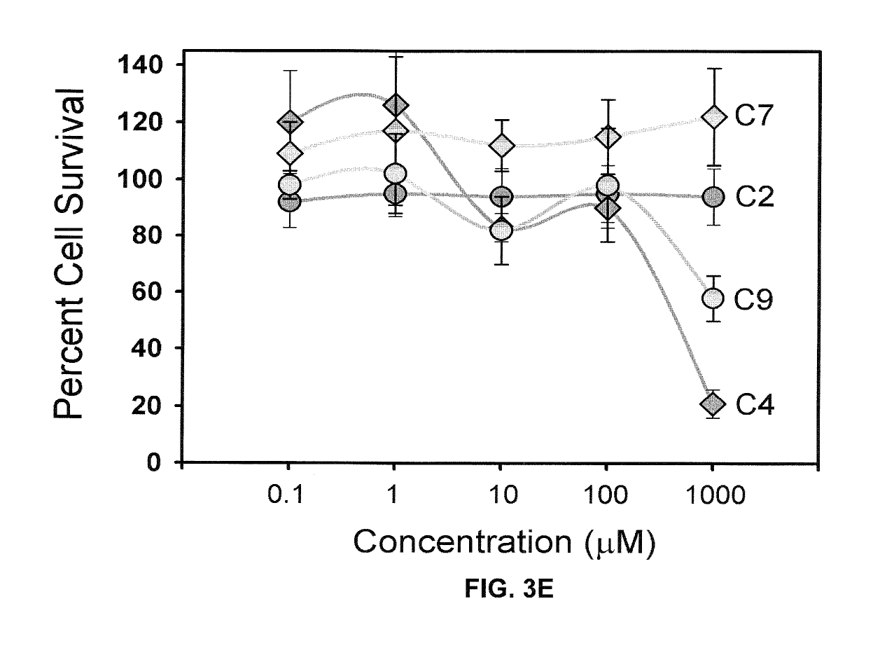

Cell survival is suppressed by Bcl2-targeted compounds: Next, we examined the effects of the Bcl-xl targeted compounds C2, C4, C7, and C9 on autophagy and cell death in Her2/Neu-positive NeuT breast carcinoma cells (FIGS. 3A-E). We found that C2 did not induce autophagy (FIG. 2D) or cell death (FIG. 3E). In addition, we report that C4 and C9 enhanced autophagy (FIGS. 3E and 3D and promoted cell death. Finally, C7 induced autophagy (data not shown), but not cell death (FIG. 3E).

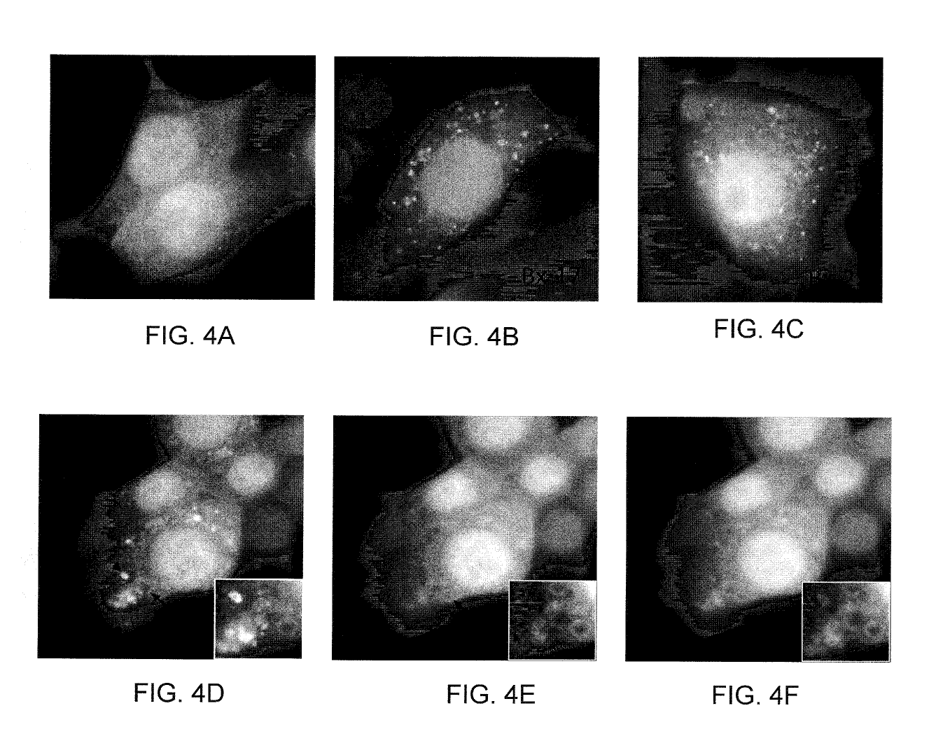

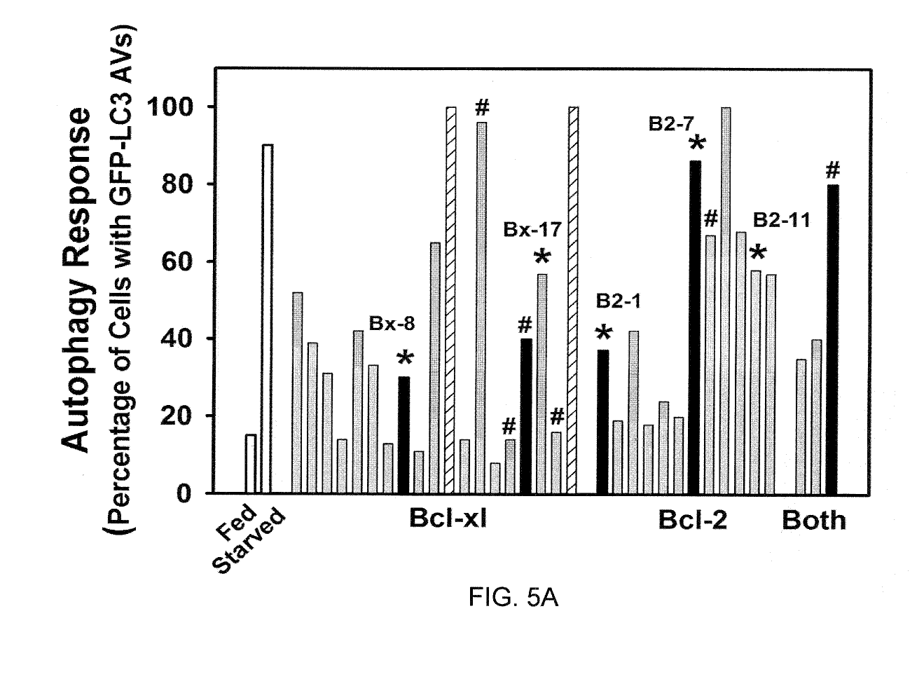

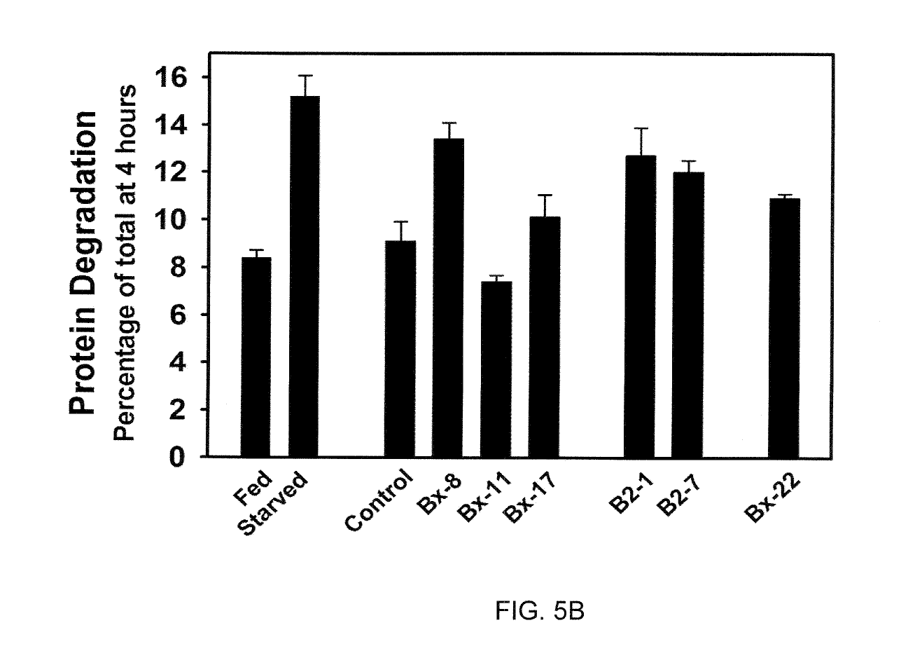

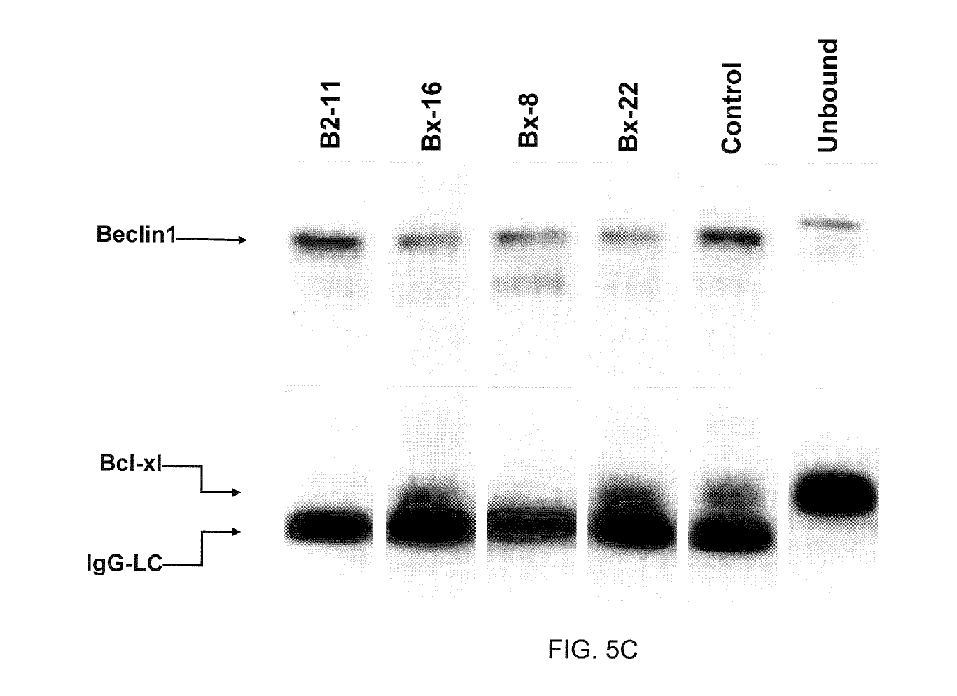

The compounds of the NCl library are ranked by in silico docking and then screened for their efficacy to activate autophagy by visualizing the cellular localization of GFP-LC3 (FIG. 4A-F) and by measuring protein turnover by pulse-chase methods (FIG. 5A-C). When autophagy is suppressed, GFP-LC3 distributes uniformly throughout the cellular cytoplasm (FIG. 4A). Autophagy response can be observed by visualizing the punctuate pattern of GFP-LC3 indicative of the presence of autophagic vacuoles. We can also use tandem-labeled LC3, RFP-GFP-LC3 to better assess the dynamics of the autophagy response by visualizing both autophagosomes and autolysosomes (FIG. 4D-4F). For example, when cells are treated with compound B2-1 (NSC 43537), both autophagosomes (arrow) and autolysosomes (arrowhead) are present suggesting that autophagy flux is enhanced instead of an intermediate blockage of the pathway. To further substantiate that the accumulation of GFP-LC3 structures is not due to a blockage of basal autophagy but the positive effects of the compounds on the autophagy response, the autophagy response is validated by quantitative protein turnover measures using radiolabeled amino acids (FIG. 5B). Finally, we determine whether the compound interferes with Beclin 1 binding to Bcl-xl (FIG. 5C) and Bcl-2 as we would suspect if the compound acts through the intended novel mechanism of action.

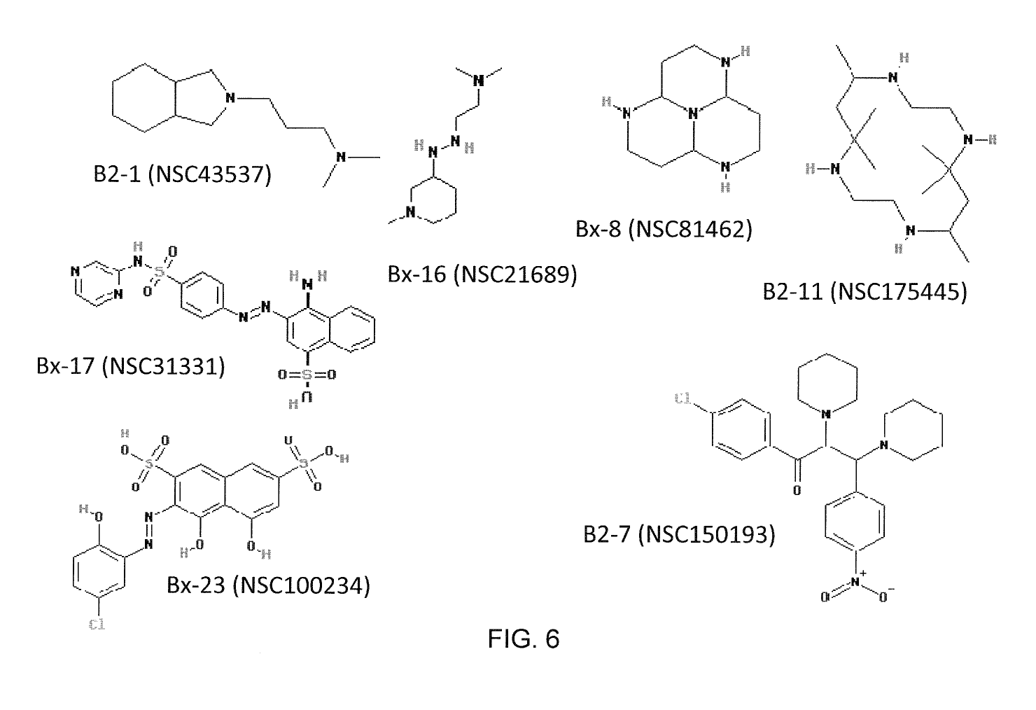

We have identified a number of compounds that stimulate autophagy, but not protein degradation while others fail to interfere with Beclin 1 binding. Bx-11 (NSC17764) and Bx19 (NSC635366) appear to promote the accumulation of autophagic vacuoles, but inhibit protein degradation. Bx-16 (NSC21689) and Bx-22 (NSC94668) activate autophagy and protein degradation, but do not appear to affect Beclin 1 binding suggesting that these compounds acts at a site different than Bcl. Nevertheless, we have identified four Bcl-targeted pro-Beclin 1 compounds, Bx-8 (NSC81462), Bx-23 (NSC100234), B2-1 (NSC43537), and B2-7 (NSC150193), that stimulate autophagy and protein degradation, while inhibiting Beclin 1 binding to Bcl proteins (see Table 1). Two additional compounds, Bx-17 (NSC31331) and B2-11 (NSC175445) should also be mentioned in that they have been shown to stimulate autophagy and interfere with Beclin 1 binding (FIG. 5A and Table 1). Finally, Bx-16 (NSC21689) is structurally similar to B2-1 (NSC43537) (FIG. 6) and activates autophagy and protein degradation (FIG. 5A). However, this compound does not inhibit Beclin 1 binding as well as Bx-8 (FIG. 5C). The chemical structures for the seven (7) pro-Beclin 1 lead compounds are shown in FIG. 6. There appear to be structural similarities between Bx-17 and Bx-23, B2-1 and Bx-16, and Bx-8 and B2-11.

TABLE 1
Bcl-targeted compounds*

| | | | | | | Activate | Stimulate | Inhibit |
|---|---|---|---|---|---|---|---|---|
| Compound | Old I.D. | New I.D. | | | | Autophagy | Protein | Beclin 1 |
| Identifier | Numbers | Numbers | Bcl-xl | Bcl-2 | Site | Response | Degradation | Binding |
| NSC17764 | C5 | Bx-11 | X | | 1 | Yes | No | No |
| NSC656252 | | | X | | 1 | Yes | | |
| NSC166100 | C3 | | X | | 1 | Yes | | No |
| NSC36400 | C4 | | X | | 1 | Yes | | No |
| NSC81462 | | Bx-8 | X | | 1 | Yes | Yes | Yes |
| NSC21689 | | Bx-16 | X | | 1 | Yes | Yes | No |
| NSC280057 | C10 | | X | | 2 | Yes | | No |
| NSC635366 | C8 | Bx-19 | X | | 2 | Yes | No | |
| NSC11693 | | | X | | 2 | Yes | | |
| NSC31331 | C7 | Bx-17 | X | | 2 | Yes | | Yes |
| NSC100234 | | Bx-23 | X | | 2 | Yes | Yes | Yes |
| NSC169468 | C12 | | | X | 2 | Yes | | |
| NSC43537 | | B2-1 | | X | 2 | Yes | Yes | Yes |
| NSC155877 | | | | X | 2 | Yes | | No |
| NSC175445 | C13 | B2-11 | | X | 2 | Yes | | Yes |
| NSC150193 | | B2-7 | | X | 2 | Yes | Yes | Yes |
| NSC94668 | C9 | Bx-22 | X | X | 2 | Yes | Yes | No |

TABLE 2

| Compound | | |
|---|---|---|
| Identifier | | Chemical Name |
| NSC 17764 | C5 | Elsix, Triscol, Triocil, Hextril, Hexoral, Glypesin, Sterisil, |
| Induces | | Oraldene, Sterilate, Hexetidine |
| autophagy; | | 5-pyrimidinamine, 1,3-bis(2-ethylhexyl)hexhydro-5- |
| causes cell death | | methyl- |
| NSC 656252 Induces autophagy; causes cell death | | 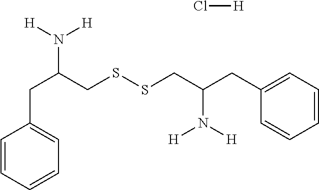 |
| NSC 166100 | C3 | Prospidin, Prospidine, Prospidium |
| Induces | | 3,12-Bis(3-chloro-2-hydroxypropyl)-3,12-diaza-6,9- |
| autophagy; not | | diazoniadispiro[5.2.5.2]hexadecane dichloride |
| tested for the | | |
| induction of cell | | |
| death | | |
| NSC 36400 Induces autophagy; causes cell death | C4 | 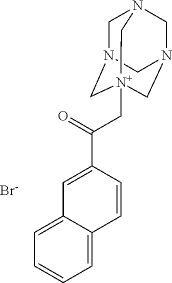 |
| NSC 280057 Induces autophagy; causes cell death | C10 | 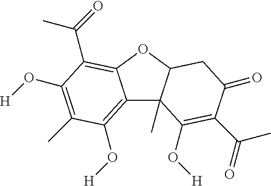 |
| NSC 635366 Induces autophagy; causes cell death | C8 | 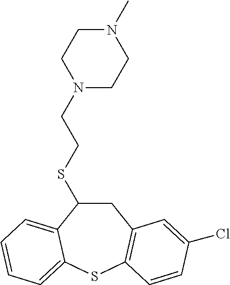 |
| NSC 11693 Induces autophagy; not tested for the induction of cell death | | 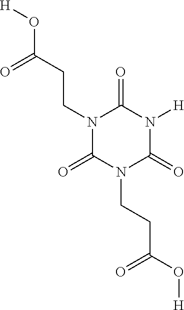 |
| NSC 31331 Induces autophagy; does not cause cell death | C7 | 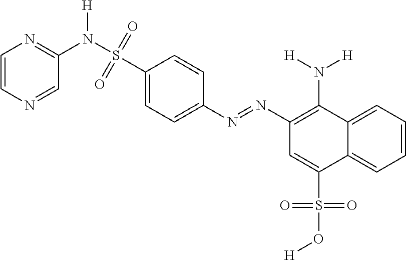 |
| NSC 169468 Induces autophagy; not tested for the induction of cell death | C12 | 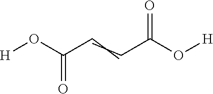 |
| | | 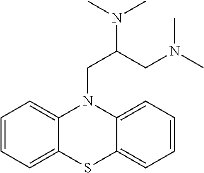 |
| NSC 43537 Induces autophagy; not tested for the induction of cell death | | 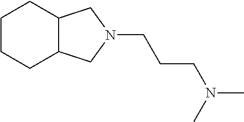 |
| | | 3-(1,3,3a,4,5,6,7,7a-octahydroisoindol-2-yl)-N,N- |
| | | dimethylpropan-1-amine |
| NSC 155877 | | 3,5,7-Triaza-1-axoniaadamantane, 1-(2,4,5- |
| | | trichlorobenzyl)-, chloride |
| NSC 175445 Induces autophagy; not tested for the induction of cell death | C13 | 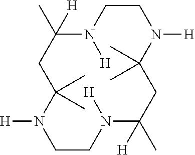 |
| | | 5,5,7,12,12,14-hexamethyl-1,4,8,11-tetrazacyclotetradecane |
| NSC 150193 Induces autophagy; not tested for the induction of cell death | | 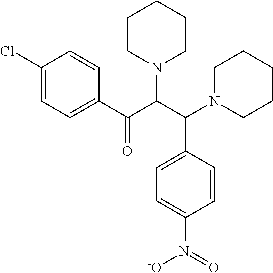 |
| | | 1-(4-chlorophenyl)-3-(4-nitrophenyl)-2,3-di(piperidin- |
| | | 1-yl)propan-1-one |
| NSC 94668 Induces autophagy; causes cell death | C9 | 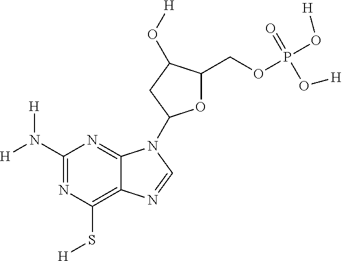 |
| NSC 81462 | | 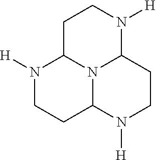 |
| | | Dodecahydro-1,4,7,9b-tetraazaphenalin; Dodecahydro- |
| | | 1,4,7,9b-tetraazaphenalene |
| NSC 21689 | | 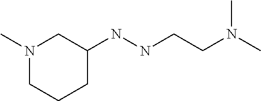 |
| NSC 100234 | | 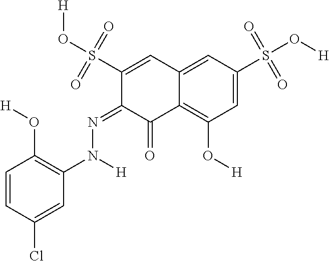 |
| | | (3E)-3-[(5-chloro-2-hydroxyphenyl)hydrazinyleidene]-5- |
| | | hydroxy-4-oxonapthalene-2,7-disulfonic acid |

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1-40. (canceled)

41. A method of increasing an individual's responsiveness to a cancer therapy comprising inhibiting the interaction of Bcl-2 and/or Bcl-XL with Beclin-1 in a cell currently undergoing cancer therapy, said method comprising the administration of a composition comprising a compound set forth in Table 1 to said individual having cancer, alone or in combination with a cancer therapy.

42. The method according to claim 41 wherein the cancer is breast cancer, pancreatic cancer, prostate cancer, gynecological cancer, skin cancer, brain cancer, neuroblastoma, glioma, a solid tumor, a hematologic malignancy, head or neck cancer, ganglioneuroma, infiltrating ductal carcinoma of the breast, adenocarcinoma of the lung, pancreatic adenocarcinoma, pancreatic islet cell tumor, liver cancer, gastric cancer, bladder cancer, colon cancer, prostate cancer, lung cancer or nasopharyngeal carcinoma.

43. The method according to claim 41, wherein said composition comprises a compound selected from dodecahydro-1,4,7,9b-tetraazaphenalene, 1-(4-chlorophenyl)-3-(4-nitrophenyl)-2,3-di(piperidin-1-yl)propan-1-one, (3E)-3-[(5-chloro-2-hydroxyphenyl)hydrazinyleidene]-5-hydroxy-4-oxonapthalene-2,7-disulfonic acid or 3-(1,3,3a,4,5,6,7,7a-octahydroisoindol-2-yl)-N,N-dimethylpropan-1-amine,5,5,7,12,12,14-hexamethyl-1,4,8,11-tetrazacyclotetradecane, NSC2 1689 or combinations thereof.

44. A method of treating cancer comprising administering an effective amount of a composition comprising a compound set forth in Table 1 alone or in combination with a cancer therapy to an individual having cancer.

45. The method according to claim 44, wherein the cancer is breast cancer, pancreatic cancer, prostate cancer, gynecological cancer, skin cancer, brain cancer, neuroblastoma, glioma, a solid tumor, a hematologic malignancy, head or neck cancer, ganglioneuroma, infiltrating ductal carcinoma of the breast, adenocarcinoma of the lung, pancreatic adenocarcinoma, pancreatic islet cell tumor, liver cancer, gastric cancer, bladder cancer, colon cancer, prostate cancer, lung cancer or nasopharyngeal carcinoma.

46. The method according to claim 44, wherein said composition comprises a compound is selected from dodecahydro-1,4,7,9b-tetraazaphenalene, 1-(4-chlorophenyl)-3-(4-nitrophenyl)-2,3-di(piperidin-1-yl)propan-1-one, (3E)-3-[(5-chloro-2-hydroxyphenyl)hydrazinyleidene]-5-hydroxy-4-oxonapthalene-2,7-disulfonic acid or (1,3,3a,4,5,6,7,7a-octahydroisoindol-2-yl)-N,N-dimethylpropan-1-amine, 5,5,7,12,12,14-hexamethyl-1,4,8,11-tetrazacyclotetradecane, NS C21689 or combinations thereof.

47. A method of treating a bacterial or viral infection comprising treating an individual infected with a bacterium or virus comprising the administration of a composition comprising one or more compound set forth in Table 1, alone or in combination with a therapeutic agent, to an infected individual.

48. The method according to claim 47, wherein said method comprises:
a) the administration of a therapeutic agent in combination with said composition to said infected individual; or
b) the administration of a composition comprising one or more compound set forth in Table 1 to said infected individual.

49. A composition comprising one or more therapeutic or chemotherapeutic agent and a compound or combination of compounds set forth in Table 1.

50. The composition according to claim 49, wherein said therapeutic or chemotherapeutic agent is selected from ciprofloxacin, norfloxacin, ofloxacin, ceftriaxone, azithromycin, nalidixic acid, ampicillin, a quinolone, a fluoroquinolone, tetracycline, a macrolide, a sulfa, clindamycin, penicillin, gentamycin, vancomycin, cefalexin, clarithyromycin, crythromycin, telithromycin, chloramphenicol, trimethoprim-sulfamethoxazole, rifamycin, isonazid, doxycycline, oxacillin, methicillin, cephalosporin, carbapenems, an anthracycline, an alkylating agent, an alkyl sulfonate, an aziridine, an ethylenimine, methylmelamine, a nitrogen mustard, a nitrosourea, an antibiotic, an antimetabolite, angiogenesis inhibitors, a folic acid analogue, a purine analogue, a pyrimidine analogue, an enzyme, a podophyllotoxin, a platinum-containing agent, a monoclonal antibody or a cytokine.

51. The composition according to claim 49, said composition comprising one or more therapeutic or chemotherapeutic agent and a compound or combination of compounds selected from dodeeahydro-1,4,7,9b-tetraazaphenalene, 1-(4-chlorophenyl)-3-(4-nitrophenyl)-2,3 di(piperidin-1-yl)propan-1-one, (3E)-3-[(5-chloro-2-hydroxyphenyl)hydrazinyleidene]-5-hydroxy-4-oxonapthalene-2,7-disulfonic acid or 3-(1,3,3a,4,5,6,7,7a-octahydroisoindol-2-yl)-N,N-dimethylpropan-1-amine.

52. A composition comprising a pharmaceutically acceptable diluent and a combination of compounds set forth in Table 1.

53. A method of decreasing the amount of protein aggregate in a cell comprising contacting a cell with a compound, or combination of compounds, set forth in Table 1 in an amount sufficient to induce autophagy in said cell and cause the degradation of the protein aggregate within the cell.

54. The method according to claim 53, wherein said cell is contacted with a compound that is not toxic to said cell.

55. The method according to claim 53, wherein said cell has a condition, syndrome or disease selected from Huntington's disease, Parkinson's disease. Charcot-Marie-Tooth type 1A syndrome, or Amyotrophic Lateral Sclerosis (ALS).

56. The method according to claim 53, wherein said compound is administered to au individual having Huntington's disease, Parkinson's disease, Charcot-Marie-Tooth type 1A syndrome, or Amyotrophic Lateral Sclerosis (ALS).

57. A method of screening compounds that stimulate and enhance the formation of autophagic vacuoles comprising:
a) scoring compounds for the potential to bind to Bcl-2 and/or Bcl-XL at Site 1 pocket (Q99, A100, G101, D102, D103, F104, and V148 of Bcl-2 and E92, A93, G94, D95, E96. F97, and V141 of Bcl-XL) and/or Site 2 pocket (V133, V134, L137, F138, N143, G145, R146, I147, and A149 of Bcl-2 and F105, D107, L108, L130, G138, R139, I140, A142, F143, F144, and S145 of Bcl-XL);
b) selecting those compounds identified as having the potential to bind to Site 1 pocket and/or Site 2 pocket of Bcl-2 and/or Bcl-XL; and
c) testing the selected compounds for the ability to enhance autophagy, said testing comprising contacting cells with at least one of the selected compounds and assessing the ability of said at least one selected compound for the ability to increase autophagy activity, wherein an increase in the formation of autophagic vacuoles is indicative of a compound that increases autophagy in said cells.
